(19) United States
(12) Patent Application Publication
(10) Pub. No.: US 2025/0003273 A1
(43) Pub. Date: Jan. 2, 2025

(54) HINGE FOR WINDOW OR DOOR

(71) Applicant: ROTO FRANK FENSTER-UND TÜRTECHNOLOGIE GMBH, Leinfelden-Eehterdingen (DE)

(72) Inventors: Hannes TRUMMER, Pirching (AT); Robert HOPFER, Hassmannstaetten (AT); Andreas SCHMIDT, Maria Lankowitz (AT); Peter RIZNIK, Tillmitsch (AT)

(21) Appl. No.: 18/694,963

(22) PCT Filed: Feb. 21, 2022
(86) PCT No.: PCT/EP2022/076169

§ 371 Date: Mar. 23, 2024

(30) Foreign Application Priority Data
Oct. 1, 2021 (AT) 50204/2021
Oct. 1, 2021 (AT) GM 50204/2021

(51) Int. Cl. E05D 7/04; E05D 3/02

(52) U.S. Cl. CPC E05D 7/0423; E05D 3/02; E05D 2007/0469

(57) ABSTRACT

The invention relates to a hinge for a door or a window, in particular designed as a roller hinge, comprising a frame hinge part with a mounting flange and a substantially cylindrical bearing, a panel hinge part (1) with a mounting flange (2) and a hinge tab (3) as well as an axle (4) pivotably connecting the two hinge parts one above the other in the vertical direction in the mounted state, with an axis pin (5) insertable into the bearing and a section with a seat (6) for the hinge tab (3) and the latter is adjustable transversely to the longitudinal axis of the axle (4) via an adjuster (7) within the seat (6). A mounting projection (8) is in the seat (6). A mounting cavity (9) is provided on the hinge tab (3) formed on the lower end face (10) of the hinge tab (3) or as an undercut cavity on the end face (11) of the hinge tab (3) facing away from the mounting flange (2) of the panel hinge part (1). In the assembled state, the mounting projection (8) comes to rest in the mounting cavity (9). A fastener (12) is provided in the axle (4) that secures the hinge tab (3) within the seat (6) in the assembled state.

### TECHNICAL FIELD

The invention relates to a hinge for a door or a window, in particular designed as a roller hinge, comprising a frame hinge part mountable in a fixed frame and having a mounting flange and a substantially cylindrical bearing, a panel hinge part mountable on the panel of the door or window and having a mounting flange and a hinge tab, and an axle that pivotably connects the two hinge parts arranged one above the other in the vertical direction in the mounted state and having an axis pin that can be inserted into the bearing and a section at the opposite end having a seat for the hinge tab, the hinge tab being adjustable transversely to the longitudinal axis of the axle via an adjuster within the seat in the axle.

### STATE OF THE ART

A door hinge designed as a roller hinge comprises a frame hinge part and a panel hinge part and both hinge parts each have a mounting flange and a section connected to an axle. The mounting flanges can be screwed to the frame and panel inside the door rebate, leaving only the axle and the mounts of the axle visible from the outside as a so-called “roller”. Installation as a visible hinge on the panel is also possible. It will be understood by those skilled in the art that the terms frame hinge part and panel hinge part do not necessarily only include the installation state in which the frame hinge part is mounted on the frame and the panel hinge part is mounted on the panel. Depending on the application, these two parts can also be installed the other way round or can also serve as a hinge between two leaves in the case of multipanel or divided doors arranged in a row.

In order to make a fine adjustment of a panel relative to the frame, it is also desirable to provide adjusters within the door hinge. In the simplest case, the adjusting screws for the position of the panel relative to the frame are located in the area of the mounting flanges of the panel hinge part or frame hinge part. This has the disadvantage that adjustment can only be carried out when the panel is open, while the correct position is checked when the panel is closed. In addition, the provision of the adjusters in the area of the mounting flanges requires more space, which means that corresponding cavities must be provided inside the door rebate, which leads to higher costs in the manufacture of these components and, depending on the application, is not feasible everywhere. In the case of hinges mounted visibly on the panel, the mounting flange cannot be designed to be particularly flat and thus visually appealing due to the adjusters provided there either.

When installing the panel, the frame hinge and the panel hinge are usually preassembled on the corresponding parts and when inserting the panel, the axis pin must then be threaded into the bearing from above by lifting the panel. This can be problematic in installation situations with a low height to the surrounding door or window frame or even prevent the use of conventional roller hinges.

### DESCRIPTION OF THE INVENTION

The object of the present invention is to improve a hinge, in particular a roller hinge, in such a way that any adjustment options are provided completely within the “roller” of the hinge. The lateral adjustment option should not result in any optical axial misalignment between the panel hinge part and the frame hinge part. The stability within the hinge should remain high. It should also be possible to insert a short panel the surrounding frame so that the hinge can be used anywhere. The manufacturing costs should be kept low, in particular because the design is kept simple and the hinge in question can be used for both left-hinged and right-hinged doors.

This object is attained in accordance with the invention in that a mounting projection in the seat of the axle is provided and a mounting cavity is provided on the hinge tab formed as a cavity on the lower end face of the hinge tab or as an undercut cavity on the end face of the hinge tab facing away from the mounting flange of the panel hinge part, and in the mounted state the mounting projection fits in the mounting cavity, and furthermore a fastener mounted in the axle is provided that secures the hinge tab within the seat in the axle in the mounted state. This design makes it possible to thread the panel hinge part laterally into the preassembled frame hinge part and axle by means of the hinge tab. To do this, the panel only needs to be lifted slightly in order to insert the hinge tab into the cavity in the axle and lift it over the mounting projection. The panel can then be lowered, allowing the mounting projection to penetrate into the mounting cavity in the hinge tab and holding the panel in place for the further assembly steps. Finally, the fastener can be used to finally secure the panel to the hinge.

According to one possible embodiment, it is advantageously provided that the seat in the axle is formed from two mounting surfaces extending perpendicular to one another and a first lower horizontal mounting surface has the mounting projection in the form of a rib aligned parallel to the second vertical mounting surface, and the fastener in the form of at least one fastening screw is provided on the second vertical mounting surface, which screw co-operates with a threaded bore mounted in the hinge tab or with a clamping piece mounted in a guide in the hinge tab in order to fix the hinge tab to the seat in the axle. This embodiment enables a very efficient transmission of force between the hinge tab and the seat. The end of the hinge tab that comes into contact with the vertical mounting surface of the seat can be thickened accordingly so that it corresponds approximately to half a circular cylinder. The thickened section of the hinge tab is slightly narrower laterally than the vertical mounting surface, so that lateral adjustment is possible.

It is a further preferred feature that the adjuster for adjusting the hinge tab in the direction transverse to the longitudinal axis of the axle is designed as an eccentric element on the second vertical mounting surface. This represents a particularly simple adjustment option for lateral adjustment. The eccentric element can already be premounted on the axle and protrudes with an extension from the vertical mounting surface. A vertical elongated hole is provided at the corresponding point on the hinge tab, into which the extension plunges when the panel is inserted. For adjustment in the lateral direction, the eccentric element is rotated, whereby the hinge tab can be displaced along the rib-shaped mounting projection formed on the horizontal mounting surface.

According to an alternative embodiment, it is an advantageous feature that the seat in the axle is formed as a blind hole or as a slot. In these embodiments, the hinge tab is essentially flat, which enables more cost-effective manufacturing. The thickness of the hinge tab is less than the width of the seat in the axle by the adjustment path.

It is also a preferred feature of a further embodiment that the mounting projection is designed as a rib or support surface that projects transversely through the seat in the axle and is on the lower inner wall in the seat and the fastener is formed by a screw extending parallel to an axis of the axle and that, in the mounted state of the hinge tab, projects from above into a complementary upper-end cavity on the hinge tab and thus secures the latter in its position. This design enables favorable force transmission with cost-effective production. The upper edge cavity for the fastener can be formed in the same way as the mounting cavity formed on the lower end face of the hinge tab, which means that the hinge tab can be mounted on both left-hinged and right-hinged doors without the need for additional different parts.

According to an alternative preferred embodiment, it is provided that the mounting projection is formed as a bar or bolt projecting transversely through the seat in the axle and the bar or bolt is preferably substantially centrally within the seat in the axle, and the fastener in the form of a fastening screw extends in the direction of insertion of the hinge tab and, in the mounted state, bears against the hinge tab or, if applicable, against a latching formation formed on the end face of the hinge tab facing away from the mounting flange of the panel hinge part. This represents a further possible embodiment characterized by low manufacturing costs, good force transmission and easy handling.

In a further preferred embodiment with a further adjustment option for the contact pressure, it is provided that the mounting projection is designed as an element movably mounted in the seat in the axle and that can be adjusted within the seat via an adjuster in the insertion direction of the hinge tab. This embodiment makes it possible in a simple manner to also create an adjustment option for the contact pressure, in that the mounting projection and thus also the hinge tab can be displaced with respect to its penetration depth in the seat in the axle.

According to a further preferred feature, it is further provided that the mounting cavity or the mounting cavities in the hinge tab are mirror-symmetrically about the horizontal central axis of the hinge tab. This has the advantage that only a few parts need to be manufactured and stored. There can also be no incorrect deliveries when ordering, as all parts can be used for both left- and right-hinged doors.

Finally, it is a further preferred feature that a height adjuster is provided formed by an adjusting screw extending axially in the frame hinge part for axially moving the bearing within the frame hinge part or the axis pin of the axle within the bearing. This makes it possible to accommodate adjustment options in all three spatial directions within the “roller” of the hinge in a space-saving manner. Any gap that may occur between the frame hinge part and the panel hinge part during height adjustment can be visually concealed by a sleeve that can be placed on the upper end of the axle.

### BRIEF DESCRIPTION OF THE DRAWINGS

The invention will now be described in greater detail with reference to examples of embodiments and with the aid of the accompanying figures. The figures show

FIG. 1 a schematic perspective view of a first embodiment of a panel hinge part and axle in the assembled state,

FIG. 2 a schematic perspective view of the panel hinge part of the embodiment according to FIG. 1,

FIG. 3 a schematic perspective view of the axle of the embodiment according to FIG. 1,

FIG. 4 a schematic perspective sectional view of a further embodiment of a panel hinge part and axle,

FIGS. 5 to 7 schematic perspective views of further possible embodiments of a panel hinge part and axle.

### WAY(S) OF CARRYING OUT THE INVENTION

In the figures only the parts of the panel hinge part 1 and the axle 4 relevant to the invention are shown. The frame hinge part with the bearing and any height adjuster is well known in the prior art and is therefore not described in more detail. The panel hinge part 1 comprises a mounting flange 2 for mounting on the panel of a door or window, and a hinge tab 3. The axle 4 has an axis pin 5 at the lower end mounted in the axis bush of an unillustrated frame hinge part. The seat 6 for the hinge tab 3 of the panel hinge part 1 is located at the upper end of the axle 4.

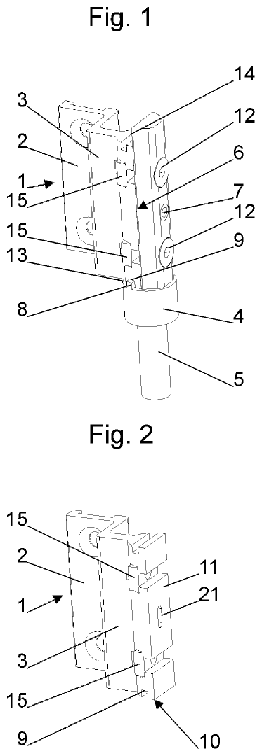

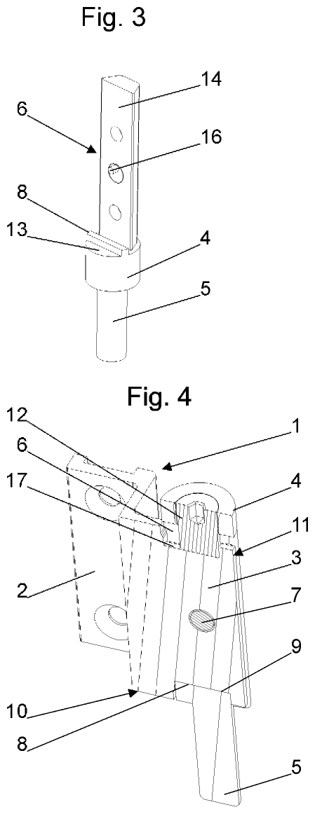

FIGS. 1 to 3 show a first possible embodiment of a hinge according to the invention. Here, the seat 6 is formed by two mounting surfaces 13, 14 standing essentially at right angles to one another. The lower horizontal mounting surface 13 has a mounting projection 8 in the form of a rib aligned parallel to the second vertical mounting surface 14, as can be seen in particular in FIG. 3.

When the panel is hung, the preassembled panel hinge part 1 with the hinge tab 3 is lifted over the mounting projection 8 and moved with the free end of the hinge tab 3 in the direction of the second vertical mounting surface 14 until it rests against it. The panel is then lowered so that the mounting projection 8 penetrates into the mounting cavity 9 located on the lower end face of the hinge tab 3. Clamping pieces 15 also extend in respective undercut grooves in the hinge tab 3. The fastener 12 in the form of fastening screws is inserted into the axle 4 and the fastening screws engage in the clamping pieces 15, whereby the hinge tab 3 is secured to the axle 4. The adjuster 7 for the lateral adjustment of the panel is in the form of an eccentric element 16 (see FIG. 3) mounted in the axle 4 and protrudes with an extension from the second vertical mounting surface 14. At the corresponding point on the hinge tab 3 there is an elongated hole 21 (see FIG. 2), into which the extension of the eccentric element 16 engages. When the fastener 12 is loosened, the hinge tab and thus the entire panel can be moved laterally by the clamping pieces 15 and the mounting projection 8. Once the panel is correctly positioned, the fastener 12 is tightened again.

The mounting cavity 9 is also provided mirror-symmetrically on the upper end face of the hinge tab 3, so that the panel hinge part 1 can be used for both left-hinged and right-hinged doors. The fastener 12 and adjuster 7 are also arranged symmetrically.

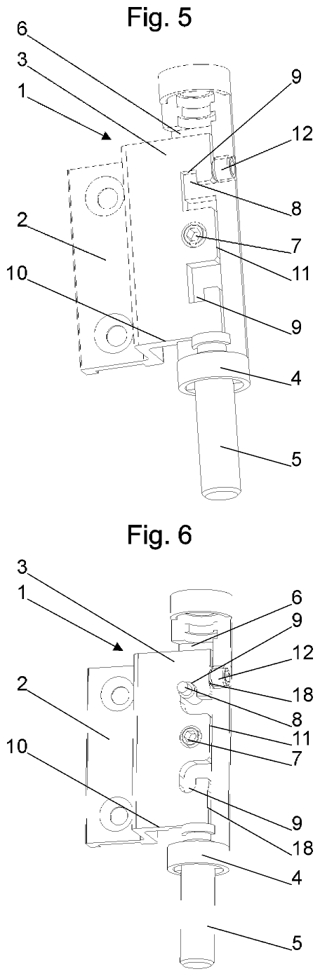

FIG. 4 shows an alternative preferred embodiment of a panel hinge part 1 and axle 4 in section. The seat 6 in the axle 4 is formed here as a slot and the hinge tab 3 is essentially flat. The thickness of the hinge tab 3 is thinner than the width of the seat 6 by the adjustment path of the lateral adjuster 7 that also applies to the following embodiments according to FIGS. 5 to 7.

In the embodiment according to FIG. 4, the lower mounting surface within the seat 6 forms the mounting projection 8 that co-operates with a complementary mounting cavity 9 on the lower end face 10 of the hinge tab 3. An upper edge cavity 17 on the hinge tab 3 forms the seat for the hinge tab fastener 12 designed here in the form of an axially extending fastening screw. As the hinge tab 3 is also symmetrical in this case, the upper edge cavity 17 can also serve as a mounting cavity 9 when the door is hinged the other way round and vice versa. The lateral adjuster 7 is formed by a screw in the hinge tab 3 supported within the seat 6.

In the following embodiments, the upper part of the axles comprises two half-shell components respectively, whereby only one half-shell is shown respectively for a better view of the mode of operation.

In the embodiment according to FIG. 5, the mounting projection 8 is designed as a hook-shaped bar projecting through the mounting 6. The respective mounting cavity 9 on the end face 11 of the hinge tab 3 facing away from the mounting flange 2 is designed as an undercut groove that in turn is provided twice with mirror symmetry. A screw in the axle 4 is provided as a fastener supported on the end face 11 of the hinge tab 3 facing away from the mounting flange 2 and thus clamps the hinge tab 3 against the mounting projection 8 in the secured state.

The embodiment according to FIG. 6 is similar to that of FIG. 5, with the difference that here an inserted bolt projecting through the seat 6 of the axle 4 serves as the mounting projection 8 and the mounting cavity 9 is designed accordingly. A screw in the axle 4 also serves here as a fastener 12 that interacts with a latching formation 18 on the end face 11 of the hinge tab 3 facing away from the mounting flange 2. The fastener 12 in this embodiment, as well as in the embodiment shown in FIG. 5, clamps the section of the hinge tab 3 between the mounting projection 8 and the fixing screw, thereby preventing this area of the hinge tab 3 from deforming under the load of the panel. The latching formation 18 is also with mirror symmetry in order to be able to use the panel hinge part 1 for leaves hinged at any orientation. Together with the fastener 12, the latching formation 18 forms an anti-lift device for the panel in the assembled state.

In all the preceding embodiments according to FIGS. 1 to 6, a further adjuster can also be provided for adjusting the contact pressure between the panel and the frame. For example, as is common in the prior art, this can be done by means of an eccentric sleeve on the axis.

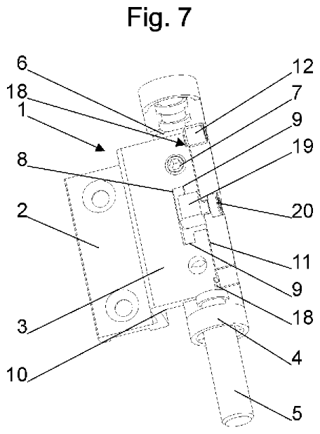

Finally, FIG. 7 shows a further embodiment in which the mounting projection 8 is designed as a movably mounted element 19 that can be displaced within the seat 6 in the direction of insertion of the hinge tab 3 by means of an adjuster 20 mounted in the axle 4. The adjuster 20 can be used together with the fastener 12 to adjust the contact pressure of the panel on the frame by adjusting the penetration depth of the hinge tab 3 in the seat in the axle 4. The hinge tab 3 can first be pulled towards the axle 4 with the adjuster 20 or pushed away from the axle 4 with the fastener 12. The hinge tab 3 can then be fixed and secured with the respective other device. This procedure can also be advantageous on a lower door hinge, for example, as the panel pushes the hinge tab 3 into the axle 4 due to its weight. This means that the adjuster 20 can also counteract any tilted positions of the panel. This embodiment also makes it possible to accommodate adjusters in all three spatial directions in a simple, cost-effective manner within the “roller” of the hinge. In this embodiment, the fastener 12 in the form of a fixing screw also interacts with a respective latching formation 18 on the hinge tab 3 and thus forms an anti-lift device for the panel in the assembled state.

All embodiments have the advantage that the panel can be mounted even if the available height is low, since in each case only the height of the mounting projection has to be overcome and the entire axis pin does not have to be threaded into the frame hinge part. The provision of all adjusters in the area of the “roller” of the hinge also has the advantage that these are accessible even when the panel is closed, and thus fine adjustment can be carried out particularly conveniently.

It will be understood by the average person skilled in the art that individual elements of the embodiments described can also be used in different combinations. For example, a latching formation 18 for the fastener 12 can also be provided in the embodiment according to FIG. 5 or a fastener according to the embodiment according to FIG. 4 can also be used in the embodiments according to FIGS. 5 to 7. A mounting projection 8, as shown in FIG. 1, can also be provided, for example, in an embodiment according to FIG. 4.

1. A roller hinge for a door or a window, the hinge comprising:
a frame hinge part mountable in a fixed frame and having a mounting flange and a substantially cylindrical bearing,
a panel hinge part mountable on a panel of a door or window and having
a mounting flange, and
a hinge tab, and
an axle that pivotably connects the two hinge parts one above the other in a mounted state and having an axis pin that can be inserted into the bearing and a section at the opposite end with a seat for the hinge tab,
an adjuster for shifting the hinge tab transversely to a longitudinal axis of the axle in the seat in the axle,
a mounting projection in the seat of the axle,
a mounting formation formed by a cavity on formed as a cavity a lower end face of the hinge tab or an undercut cavity on a face of the hinge tab directed away from the mounting flange of the panel hinge part and, in the mounted state the mounting projection, fitting in the mounting cavity, and
a fastener mounted in the axle and securing the hinge tab in the seat in the axle in the mounted state.

2. The hinge for a door or a window according to claim 1, wherein the seat in the axle is formed from first lower and second vertical mounting surfaces extending perpendicular to each other relative to one another, the first lower horizontal mounting surface carrying the mounting projection in the form of a rib aligned parallel to the second vertical mounting surface, the fastener being at least one fixing screw provided on the second vertical mounting surface and cooperating with a threaded bore mounted in the hinge tab or with a clamping piece mounted in a guide in the hinge tab in order to fix the hinge tab to the seat in the axle.

3. The hinge for a door or a window according to claim 2, wherein the adjuster for adjusting the hinge tab in the direction transverse to the longitudinal axis of the axle is designed as an eccentric element on the second vertical mounting surface.

4. The hinge for a door or a window according to claim 1, wherein the seat in the axle is formed as a blind hole or as a slot.

5. The hinge for a door or a window according to claim 4, wherein the mounting projection is formed as a rib or support surface extending transversely through the seat in the axle, the rib or support surface being on a lower inner wall in the seat and the fastener being formed by a screw parallel to an axis of the axle such that, in the mounted state of the hinge tab, the screw projects from above into a complementary upper-end cavity on the hinge tab and thus secures the latter in position on the axle.

6. The hinge for a door or a window according to claim 4, wherein the mounting projection is formed as a bar or bolt projecting transversely through the seat in the axle and the bar or bolt is located substantially centrally within the seat in the axle, and the fastener in the form of a fastening screw extends in a direction of insertion of the hinge tab and in the mounted state rests against the hinge tab or against a latching formation formed on the end face of the hinge tab facing away from the mounting flange of the panel hinge part.

7. The hinge for a door or a window according to claim 4, wherein the mounting projection is an element movably mounted in the seat of the axle and can be adjusted within the seat via an adjuster in the insertion direction of the hinge tab.

8. The hinge for a door or a window according to claim 1, wherein the mounting cavity or the mounting cavities in the hinge tab are mirror-symmetrically flank a horizontal central axis of the hinge tab.

9. The hinge for a door or a window according to claim 1, further comprising:
a height adjuster formed by an adjusting screw extending axially in the frame hinge part for axially moving the bearing within the frame hinge part or the axis pin of the axle within the bearing.
